(19) United States
(12) Patent Application Publication
(10) Pub. No.: US 2014/0183380 A1
(43) Pub. Date: Jul. 3, 2014

(54) MEASURING UNIT AND GAS ANALYZING APPARATUS

(71) Applicants: Juichiro Ukon, Kyoto-shi (JP); Takuya Ido, Kyoto-shi (JP); Toshikazu Ohnishi, Kyoto-shi (JP); Toshiyuki Tsujimoto, Kyoto-shi (JP)

(72) Inventors: Juichiro Ukon, Kyoto-shi (JP); Takuya Ido, Kyoto-shi (JP); Toshikazu Ohnishi, Kyoto-shi (JP); Toshiyuki Tsujimoto, Kyoto-shi (JP)

(73) Assignee: HORIBA, LTD., Kyoto-shi, Kyoto (JP)

(21) Appl. No.: 14/119,120

(22) PCT Filed: May 17, 2012
(86) PCT No.: PCT/JP2012/062595

§ 371 Date: Nov. 20, 2013

(30) Foreign Application Priority Data
May 20, 2011 (JP) 2011-113789

(51) Int. Cl. G01N 33/00

(52) U.S. Cl. CPC G01N 33/0009
USPC 250/573

(57) ABSTRACT

A measurement unit used in an analyzing apparatus for measuring concentrations of component gases in a sample gas comprises a light emitting unit configured to emit a measurement light to the sample gas, a light receiving unit configured to receive the measurement light on a light receiving plane, a purge air introducing unit configured to introduce a purge air into a vicinity of at least one of the light emitting unit and the light receiving unit, and a condensing lens arranged in an optical path of the measurement light from the light emitting unit to the light receiving unit, the condensing lens being configured to condense the measurement light within the light receiving plane of the light receiving unit, a propagation path of the measurement light being varied by a thermal lens effect caused by a temperature difference between the sample gas and the purge air.

### TECHNICAL FIELD

The present invention relates to a gas analyzing apparatus and a measurement unit. More specifically, the present invention relates to a gas analyzing apparatus that analyzes a concentration of a predetermined component in a sample gas using a light absorption technique, and a measurement unit used in the gas analyzing apparatus.

### BACKGROUND

A combustion exhaust gas, which is expelled from a boiler that combusts coal or heavy oil, includes gases such as NOx, SOx, CO2, CO, etc. Gas analyzing apparatuses for analyzing contents of these components in the gas have previously been developed. Various types of apparatuses have been developed, such as an open-path type apparatus and a probe-type apparatus.

### SUMMARY

One example of a cylindrical measurement unit used in the above-described probe-type gas analyzing apparatus is disclosed in Patent Citation 1. The measurement unit disclosed in Patent Citation 1 emits a measurement light from a light source that is arranged at one end side of a cylindrical casing so as to pass the measurement light through a sample gas that is introduced into the inner space of the casing. The measurement light is reflected by a reflecting mirror that is arranged at another end side of the casing and the reflected measurement light is received by a light receiving sensor. An amount of the measurement light absorbed by the sample gas is derived by subtracting one information of the measurement light from another. One information is the information that can be derived from the light receiving sensor. Another is the information of the measurement light at the time when the measurement light is emitted from the light source. Then, the concentration of the predetermined component in the sample gas can be derived based on the amount of the measurement light absorbed by the sample gas.

Due to such a measurement principle, in order to perform accurate analyses in the gas analyzing apparatus using the above-described measurement light, it is important to receive the measurement light within the light receiving plane of the light receiving sensor. From such a point of view, it is considered that the positioning of the optical components, such as the light source, the reflecting mirror, the light receiving, etc., can be performed in the probe-type gas analyzing apparatuses easier than the open-path type gas analyzing apparatus. This is because the optical components of the probe-type gas analyzing apparatus are fixed in a single casing as described above. On the other hand, these optical components of the open-path type gas analyzing apparatus are arranged separately. In other words, it is considered to be easier to set the irradiation point of the measurement light within the light receiving plane of the light receiving sensor in the probe-type gas analyzing apparatus.

Patent Citation 1: U.S. Pat. No. 6,809,825

### DISCLOSURE OF INVENTION

### Technical Problem

However, even with the above-described probe-type gas analyzing apparatus, situations arise in which the irradiation point of the measurement light cannot be set within the light receiving plane of the light receiving sensor.

In measurement units, in which the optical components such as the above-described sensor, the reflecting mirror, etc. are used, there is the case in which a cleaning air (so-called purge air) is introduced around the optical components in the casing. The cleaning air is introduced with a predetermined pressure in order to avoid the contamination of the optical components due to dust contained in the sample gas, etc.

While the temperature of the sample gas expelled from the above-described boiler is very high, the temperature of the purge air is typically the same as the ambient temperature. When there is a temperature difference between the purge air and the sample gas, spatial distribution of temperature occurs inside the casing of the measurement unit, such as on the path of the measurement light. When such a spatial distribution of temperature occurs, the spatial refractive index is changed proportionally to the spatial distribution of temperature. Then, the measurement light propagating in the space might be refracted since the change of the refractive index causes the effect equivalent to transitional optical lenses (so-called thermal lens effect).

As shown in FIG. 9, the measurement light Lb2 should propagate on a straight path R1. However, Lb2 might propagate on a refracted path, like the path R3, due to the temperature difference between the sample gas Sg and the purge air Pa.

FIG. 9 is an image view showing that the measurement light is improperly received in the conventional measurement unit. If the measurement light Lb2 is refracted in such a manner, the measurement light Lb2 cannot be received within the light receiving plane of the light receiving sensor. Therefore, it is sometimes difficult to perform an accurate analysis.

In addition, the state of the refraction of the measurement light Lb2 changes with time. This is due to the thermal lens effect changing because of changes in the flows of the sample gas Sg and the purge air Pa. As a result, even if the measurement light Lb2 is emitted onto the light receiving plane of the light receiving sensor 54, the irradiation point Lbp2 of the measurement light Lb2 on the light receiving plane may fluctuate, as shown in FIG. 10.

FIG. 10 is a view showing that the irradiation point Lbp2 fluctuates on the light receiving plane in the conventional measurement unit. In FIG. 10, a locus line Tr2 is a movement locus of the irradiation point Lbp2. In FIG. 10, since the locus line Tr2 snakes, it is shown that the irradiation point Lbp2 fluctuates as described above. When the position of the irradiation point Lbp2 moves in the light receiving sensor, stable signals might not be derived from the light receiving sensor, even if the intensity of the measurement light Lb2 is constant, because the light receiving sensitivity of the light receiving sensor may be dependent on the positions of the light receiving plane.

The measurement light might also be refracted, due to the thermal lens effect, in the open-path gas analyzing apparatus when using the purge gas in the same manner as the probe-type gas analyzing apparatus. In such a configuration, the irradiation point of the measurement light sometimes cannot be set properly within the light receiving plane of the light receiving sensor.

The present invention was conceived in light of the above-described problems and the object of the present invention is to provide the measurement unit and the gas analyzing apparatus that can analyze the sample gas more accurately than the conventional techniques.

### Technical Solution

A measurement unit, according to one aspect of the present invention, is the measurement unit that is used in an analyzing apparatus for measuring concentrations of component gases in a sample gas. The measurement unit comprises a light emitting unit, a light receiving unit, a purge air introducing unit, and a condensing lens. The light emitting unit is configured to emit a measurement light to the sample gas. The light receiving unit is configured to receive the measurement light on a light receiving plane. The purge air introducing unit is configured to introduce a purge air into a vicinity of at least one of the light emitting unit and the light receiving unit. The condensing lens is arranged in an optical path of the measurement light. The optical path of the measurement light extends from the light emitting unit to the light receiving unit. The condensing lens is configured to condense, within the light receiving plane of the light receiving unit, the measurement light. In this case, the propagation path of the measurement light is variable due to a thermal lens effect caused by a temperature difference between the sample gas and the purge air.

The measurement light can be properly received within the light receiving plane of the light receiving unit, even if the path of the measurement light is refracted due to the thermal lens effect. In addition, the measurement light can be stably emitted to the predetermined position of the light receiving plane. Therefore, the information of the measurement light can be accurately derived with the light receiving unit, and accurate analysis of the predetermined component gas, in the sample gas, can be performed based on such information.

The condensing lens may be arranged immediately in front of the light receiving unit. The measurement unit may further include an optical window arranged immediately in front of the condensing lens. The optical window is configured to protect at least the condensing lens. The purge air introducing unit may introduce the purge air immediately in front of the optical window.

Optical components can be properly protected by introducing the purge air in the appropriate position. Even if the thermal lens effect occurs when the purge air is introduced, the measurement light can properly be received by the light receiving plane of the light receiving unit by utilizing the condensing lens.

The measurement unit may further comprise a cylindrical probe tube having openings to introduce the sample gas into the probe tube. The purge air introducing unit may introduce the purge air inside the probe tube. The light emitting unit may emit the measurement light to the sample gas introduced inside the probe tube.

By utilizing the condensing lens, the measurement light can properly be received within the light receiving plane of the light receiving unit. This is especially true for a probe-type measurement unit, in which bending of the measurement light due to the thermal lens effect easily occurs.

The measurement unit may further comprise a reflection mirror arranged at one end portion of the probe tube. The light emitting unit may be arranged at another end portion of the probe tube. The light emitting unit is configured to emit the measurement light toward the reflection mirror. The light receiving unit may be arranged at the another end portion of the probe tube. The light receiving unit is configured to receive the measurement light reflected by the reflection mirror.

The numerical aperture of the condensing lens may be greater than or equal to 0.08.

With this configuration, when the light receiving plane of the light receiving unit is formed with a multi-layered semiconductor, the interference of the measurement light can be inhibited. The interference is caused by the multiple reflections of the measurement light that occur in the semiconductor layers. The intensity of the measurement light can be detected accurately with the light receiving unit.

The light receiving unit may be tilted with respect to the condensing lens so that an angle between the light receiving plane and an image formation plane of the condensing lens is 10 degrees or more.

With this configuration, when the light receiving plane of the light receiving unit is formed with a multi-layered semiconductor, the interference of the measurement light can be inhibited. The interference is caused by the multiple reflections of the measurement light that occur in the semiconductor layers. The intensity of the measurement light can be detected accurately with the light receiving unit.

### BRIEF DESCRIPTION OF THE DRAWINGS

FIG. 1 is an external view of the measurement unit 1 according to the first embodiment.

FIG. 2 is a cross-sectional view showing the inner structure of the measurement unit 1 according to the first embodiment.

FIG. 3 is an image view showing that the measurement light Lb1 refracted in the measurement unit 1 is guided within the light receiving plane of the light receiving unit 24 by the condensing lens 23.

FIG. 4 is a view showing that the movement of the irradiation point Lbp1 on the light receiving plane is inhibited in the measurement unit 1.

FIG. 5 is a cross-sectional view showing the detailed structure of the light receiving unit 24.

FIG. 6 is a graph showing the stabilities of the electrical signals of the light receiving unit 24 derived at each of settings of the condensing lens 23 and the light receiving unit 24 in the measurement unit 1 according to the first embodiment.

FIG. 7 is an enlarged view of a part of FIG. 6.

FIG. 8 is a cross-sectional view showing the inner structure of the measurement unit 2 according to the second embodiment.

FIG. 9 is an image view showing that the measurement light Lb2 is not received properly in the conventional measurement unit.

FIG. 10 is a view showing that the irradiation point Lbp2 fluctuates on the light receiving plane in the conventional measurement unit.

### DETAILED DESCRIPTION

### Embodiment

### First Embodiment

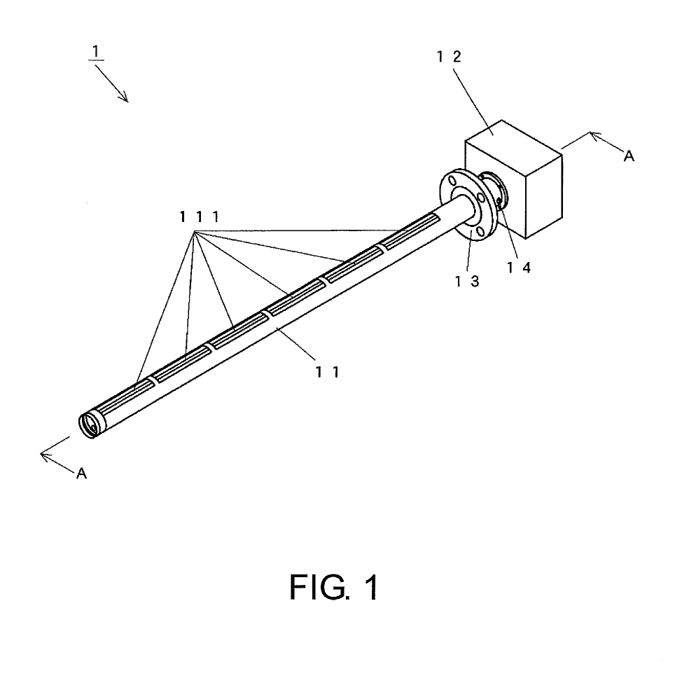

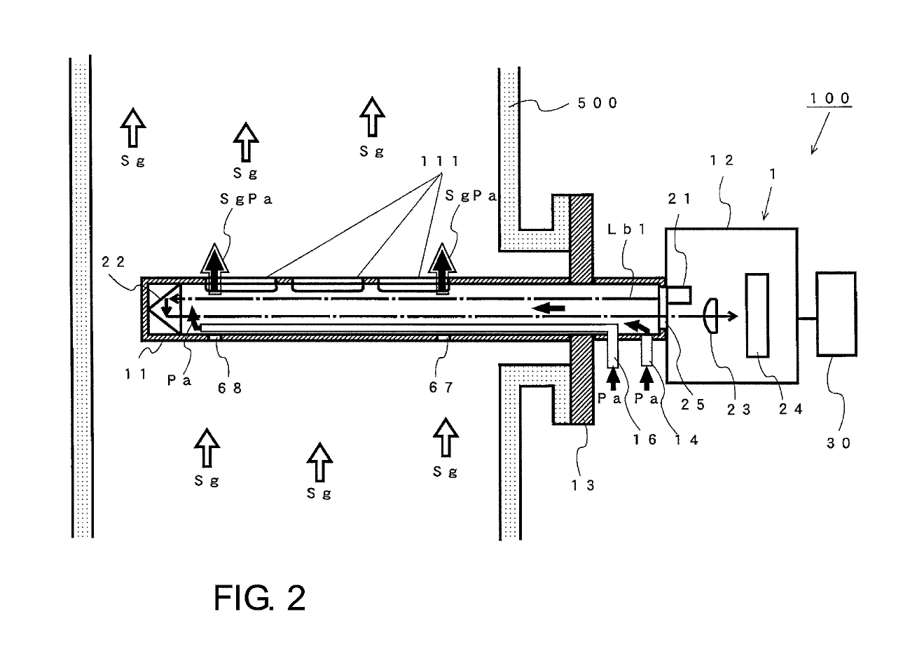

A measurement unit 1 and a gas analyzing apparatus 100 using the measurement unit 1 will be explained below. The gas analyzing apparatus 100 is a so-called probe-type gas analyzing system and the measurement unit 1 is a so-called probe unit. First, the structure of the measurement unit 1 will be explained, referring to FIG. 1 and FIG. 2. FIG. 1 is an external view of the measurement unit 1 according to the first embodiment. FIG. 2 is a cross-sectional view showing the inner structure of the measurement unit 1 according to the first embodiment. FIG. 2 is a view that includes the A-A cross section of the measurement unit 1 shown in FIG. 1. As shown in FIG. 1, the measurement unit 1 includes a probe tube 11, an optical unit 12, and a flange 13.

The probe tube 11 is a cylindrical member in which introducing openings 111 are formed. The introducing openings 111 introduce a sample gas Sg inside the probe tube 11 by diffusion of the sample gas Sg. The probe tube 11 may be made of any metallic material appropriate for the environment where the measurement unit 1 is used. As shown in FIG. 1, the introducing openings 111 are formed as intermittent slits on the side plane of the probe tube 11. As shown in FIG. 2, a reflection mirror 22 is arranged at one inner end side portion of the probe tube 11. On the other hand, the other end side portion of the probe tube 11 is connected to the optical unit 12.

As shown in FIG. 2, the optical unit 12 is the optical apparatus that includes a light emitting unit 21, a condensing lens 23, a light receiving unit 24, and an optical window 25. The light emitting unit 21 is the light source apparatus that emits a measurement light Lb1 to the inside of the probe tube 11. The light emitting unit 21 is typically a light source apparatus that emits the light with a predetermined wavelength band, such as an infrared laser oscillating apparatus, an LED (Light Emitting Diode), or a deuterium lamp that emits an ultraviolet light. The light receiving unit 24 is the light receiving apparatus that receives the measurement light Lb1 on the light receiving plane. The light receiving unit 24 is typically a photoelectric converting apparatus, such as a photodiode. The condensing lens 23 is the lens member that condenses the measurement light Lb1 within the light receiving plane of the light receiving unit 24. The condensing lens 23 is arranged immediately in front of the light receiving unit 24. The light receiving unit 24 is electrically connected to a processing apparatus 15 and sends information (for example, an intensity) of the measurement light Lb1 to the processing apparatus 30 as an electric signal. The optical window 25 is the planar member that is made of the material that transmits the measurement light Lb1. As shown in FIG. 2, the optical window 25 may be arranged at the point where the casing of the optical unit 12 and the probe tube 11 are connected. In other words, the optical window 25 may be disposed immediately in front of the light emitting unit 21 and the condensing lens 23. The optical window 25 protects the light emitting unit 21, and the condensing lens 23. It should be noted that the above-described reflection mirror 22 is arranged inside the probe tube 11 in advance, so as to reflect the measurement light Lb1. Measurement light Lb1 is emitted from the light emitting unit 21, toward the light receiving unit 24.

The processing apparatus 30 controls the operations of the light emitting unit 21 and the light receiving unit 24 and calculates the concentration of the predetermined component in the probe tube 11 based on the signal received from the light receiving unit 24. The processing apparatus 30 typically includes an information processing apparatus, such as a CPU (Central Processing Unit), etc., a storing apparatus, such as a memory, etc., an interface apparatus that receives the operations from a user, a displaying apparatus that displays results of the analysis, etc. The processing apparatus 30 performs the arithmetic processes based on the operations by the user and the program stored in the storing apparatus.

As shown in FIG. 2, in the above-described probe tube 11, a purge air introducing port 14 is arranged inside the probe tube 11, the purge air introducing port 14 introduces a purge air Pa. The purge air introducing port 14 is arranged in the vicinity of the connection part at which the probe tube 11 and the optical unit 12 are connected, as shown in FIG. 1 and FIG. 2. Arranged in such a manner, introducing the purge air Pa with the predetermined pressure from the purge air introducing port 14 prevents the sample gas Sg and dust inside the probe tube 11 from touching the optical window 25 of the optical unit 12. Therefore, the contamination and corrosion of the optical window 25 can be inhibited. The flow paths of the purge air Pa are shown in thick black arrows in FIG. 2. In addition, the flow paths of the sample gas Sg is shown in white arrows in FIG. 2. It is preferable that the above-described purge air introducing port 14 is arranged so as to introduce the purge air immediately in front of the optical window 25. The optical components such as the condensing lens 23, etc. can be properly protected by introducing the purge air Pa at such an appropriate position.

The probe tube 11 further includes a purge air introducing pipe 16 that introduces the purge air Pa in front of the reflection mirror 22 to protect the reflection mirror 22. Such a structure avoids causing the sample gas Sg, and dust in the probe tube 11, from coming into contact with the reflection mirror 22. Therefore, the contamination and corrosion of the reflection mirror 22 can be inhibited.

In addition, as shown in FIG. 2, holes 67 and 68 are formed at the both ends of the introducing openings 111, and at the opposite side of the introducing openings 111 (at the side of the upper stream of the sample gas Sg) of the probe tube 11. Flowing the sample gas Sg from these holes 67 and 68 can prevent the purge air Pa from flowing into the middle part of the probe tube 11. The purge air Pa is expelled from the introducing openings 111 (SgPa) while mixing with the sample gas Sg. The introducing openings 111 are also used as an outlet for exhausting the purge air Pa.

The flange 13 is the member that fixes the measurement unit 1 to a funnel 500 that expels the sample gas Sg or to a container that encapsulates the sample gas Sg (See FIG. 2). The flange 13 is, for example, a disk-like member and arranged so as to be passed through by the probe tube 11 at the one end side (the side connected to the optical unit) of the probe tube 11. The flange 13 is fixed to the funnel 500 with bolts, for example.

Next, the optical path of the measurement light Lb1 emitted from the light emitting unit 21 will be explained. The propagation path of the measurement light Lb1 is shown in a chain line in FIG. 2. As shown in FIG. 2, the measurement light Lb1 emitted from the light emitting unit 21 passes through the space inside the probe tube 11 and is reflected by the reflection mirror 22. The probe tube 11 is filled with the sample gas Sg. The measurement light reflected by the reflection mirror 22 passes through the space inside the probe tube 11 and propagates toward the light receiving unit 24. Thus, the measurement light Lb1 reciprocates through the space inside the probe tube and is received by the light receiving unit 24.

Here, the measurement light Lb1 reflected by the reflection mirror 22 might be refracted due to so-called thermal lens effect, and may propagate in a path different than straight from the reflection mirror 22 to the light receiving unit 24 in the probe tube 11. In more detail, the sample gas Sg and the purge air Pa flow into the probe tube 11. The spatial temperature gradient might be generated when the temperature difference between the sample gas Sg and the purge gas Pa exists. Thus, the change of the spatial refractive index might be generated in accordance with the spatial temperature gradient and therefore the measurement light Lb1 might be refracted.

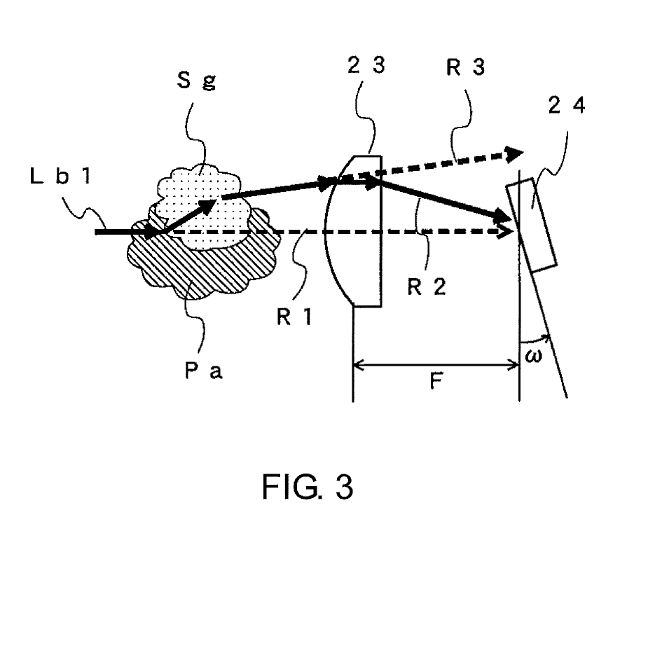

Taking this point into consideration, the measurement unit 1 includes the condensing lens 23. With the measurement unit 1 including the condensing lens 23, the measurement light Lb1 can be guided within the light receiving plane of the light receiving unit 24 by changing the propagation direction of the refracted measurement light Lb1, as shown in FIG. 3. FIG. 3 is an image view showing that the measurement light Lb1 refracted in the measurement unit 1 is guided to the light receiving plane of the light receiving unit 24 by the condensing lens 23. Specifically, as shown in FIG. 3, the measurement light Lb1, which is refracted due to the thermal lens effect, enters the condensing lens 23 and propagates on a path R2. Then, the measurement light Lb1 finally reaches within the light receiving plane of the light receiving unit 24. Without the condensing lens 23, the measurement light Lb1, which is refracted due to the thermal lens effect, propagates on a path R3.

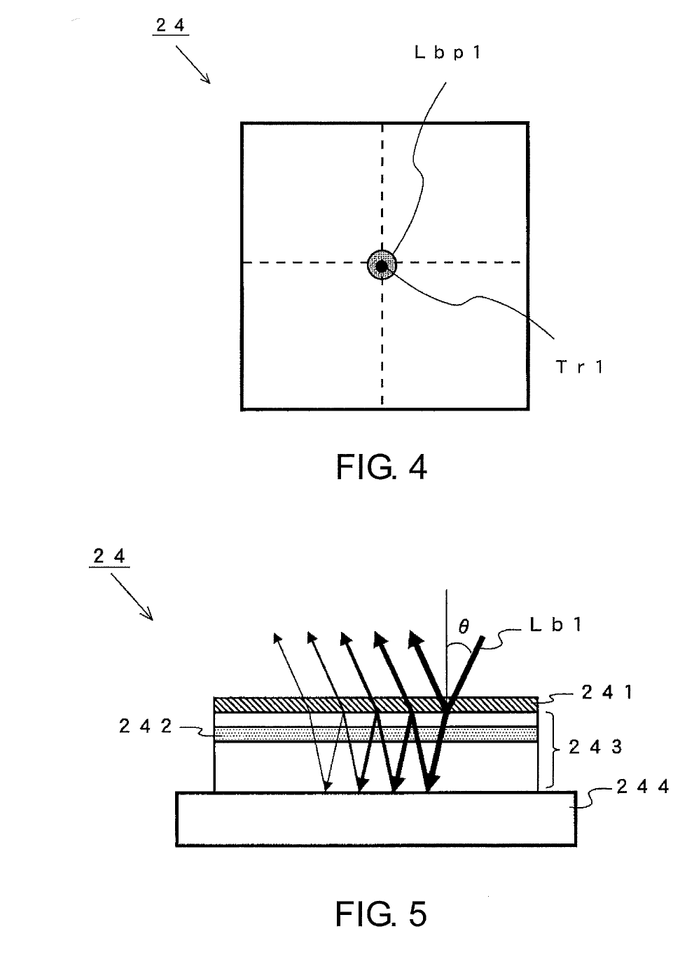

In addition, with the measurement unit 1, the measurement light Lb1 entering the condensing lens 23 is condensed to the predetermined condensing point, in accordance with the property of the condensing lens 23. Therefore, unnecessary movement of the irradiation point Lbp1 can be inhibited. The irradiation point Lbp1 is a point where the measurement light Lb1 intersects the light receiving plane of the light receiving unit 24, as shown in FIG. 4. FIG. 4 is a view showing that the movement of the irradiation point Lbp1 on the light receiving plane is inhibited in the measurement unit 1. In FIG. 4, the locus line Tr1 shows the movement locus of the irradiation point Lbp1. Since the locus line Tr1 does not snake in FIG. 4, it is shown that the movement of the irradiation point Lbp1 is inhibited as described above. Thus, with the measurement unit 1 according to the present embodiment, the measurement light Lb1 can be received within the predetermined area of the light receiving plane of the light receiving unit 24. Therefore, a stable light receiving signal can be derived even with the light receiving unit 24 that has a positional dependence of the detection sensitivity.

As described above, with the measurement unit 1, the measurement light Lb1 that has reciprocated inside the probe tube 11 can be properly received within the light receiving plane of the light receiving unit 24. By receiving the measurement light Lb1 within the light receiving plane of the light receiving unit 24, an electrical signal that corresponds to the intensity of the measurement light Lb1 can be derived. Therefore, the gas analyzing apparatus 100 comprising the measurement unit 1 can analyze the sample gas Sg accurately based on the electric signal that corresponds to the intensity of the measurement light Lb1.

In probe-type gas analyzing apparatuses, the proportion of the purge air relative to the sample gas is greater than that in the open-path type apparatus. This is because the sample gas and the purge air are introduced into a limited space inside the probe tube. In other words, the refraction of the measurement light due to the thermal lens effect is greater in a probe-type gas analyzing apparatus than in an open-path type gas analyzing apparatus. Therefore, it is effective to apply the present embodiment to the above-described probe-type measurement unit 1 and the gas analyzing apparatus 100 using the probe-type measurement unit 1.

It is preferable that the numerical aperture NA (Numerical Aperture) of the lens used as the condensing lens 23 is 0.08 or more. It is preferable that the light receiving unit 24 is arranged such that the light receiving plane of the light receiving unit 24 is substantially perpendicular to the optical axis of the condensing lens 23. The numerical aperture NA is the value expressed by the equation (1), where φ is the maximum angle of the light beam, which the condensing lens 23 condenses, relative to the optical axis of the condensing lens 23, n is the refractive index of the medium between the condensing lens 23 and the light receiving unit 24.

NA=n sin φ (1)

Namely, the numerical aperture NA is the value proportional to the condensing angle of the condensing lens 23.

In addition, the light receiving unit 24 is arranged while being tilted relative to the condensing lens so that a tilting angle ω is greater than or equal to 10 degrees, where the tilting angle ω is the angle of the light receiving plane of the light receiving unit 24 relative to the image forming plane of the condensing lens 23. Thus, since the next multiple reflections can be inhibited without increasing the numerical aperture NA to an extremely large value, the space for setting the distance between the condensing lens 23 and the light receiving unit 24 can be increased. Moreover, it can prevent the incident light from reflecting, returning, and then becoming a noise in the signal.

The reasons why it is preferable that the numerical aperture of the condensing lens 23 is greater than or equal to 0.08, and the tilting angle ω is greater than or equal to 10 degrees, will explained below.

The light receiving plane of the above-described light receiving unit 24 has multiple layers of semiconductors as shown in FIG. 5. FIG. 5 is a cross-sectional view showing the detailed structure of the light receiving unit 24. Specifically, the light receiving unit 24 includes a package substrate 244, an InP wafer layer 243 arranged on the principal surface of the package substrate 244, an InGaAs absorbing layer 242 formed in the InP wafer layer 243, and an AR (Anti Reflection) coating layer 241 formed on the surface of the InP wafer layer 243. Gold plating is formed on the surface of the package substrate 244. The plane where the AR coating layer 241 is formed is the light receiving plane of the light receiving unit 24. The measurement light Lb1 entering the light receiving surface of the light receiving unit 24 is absorbed by the InGaAs absorbing layer 242. Then, the light receiving unit 24 generates an electric signal in accordance with the intensity of the light absorbed by the InGaAs absorbing layer 242, and outputs this signal to the processing apparatus 30. Several well-known techniques can be used as the technique with which the light receiving unit 24 performs the photoelectric conversion of the measurement light Lb1.

In the conventional techniques, there has been the case in which an electric signal that corresponds to the intensity of the measurement light Lb1 cannot be derived accurately. This is because the multiple reflection of the measurement light Lb1 in the semiconductor layers shown in FIG. 5 causes the interference of the measurement light Lb1 (so-called etalon effect) when the measurement light Lb1 is received by the light receiving unit 24. In more detail, the measurement light Lb1 entering the light receiving unit 24 propagates into the InP wafer layer 243, while a part of the measurement light Lb1 is reflected by the AR coating layer 241. After a part of the measurement light Lb1 is absorbed by the InGaAs absorbing layer 242 in the InP wafer layer 243, the measurement light Lb1 transmits through these layers and is reflected at the surface of the package substrate 244. The measurement light Lb1 reflected at the surface of the package substrate 244 transmits again through the InP wafer layer 243 and the InGaAs absorbing layer 242 and is then reflected again at the interface between the AR coating layer 241 and the InP wafer layer 243. Thus, there has been the case in which, when the measurement light Lb1 enters the light receiving plane of the light receiving unit 24 at the predetermined angle of incidence, the measurement light Lb1 is repeatedly reflected in the semiconductor layers that form the light receiving unit 24. Then the reflected measurement light and the incident measurement light interfere with each other. There has also been the case in which, when such interference occurs, even if the intensity of the measurement light Lb1 is constant at the time when the measurement light Lb1 enters the light receiving unit 24, the magnitude of the electric signal derived from the light receiving unit 24 becomes unstable because the amount of the measurement light Lb1 absorbed in the InGaAs absorbing layer 242 is unstable.

Considering the above, it is preferable that the etalon effect is inhibited in the measurement unit 1. In order to achieve this, it is preferable that the multiple reflections are inhibited by increasing the angle of incidence θ of the measurement light Lb1 when the measurement light Lb1 enters the light receiving plane of the light receiving unit 24. Here, the angle of incidence θ can be large as the numerical aperture NA becomes large. In addition, the angle of incidence θ can also be adjusted by tilting the light receiving plane of the light receiving unit 24 with respect to the optical axis of the condensing lens 23. The inventor considering this point has concluded, by performing the experiments that will be described later, that the numerical aperture NA of the condensing lens 23 is preferably greater than or equal to 0.08 and further the angle of incidence θ is preferably greater than or equal to 10 degrees.

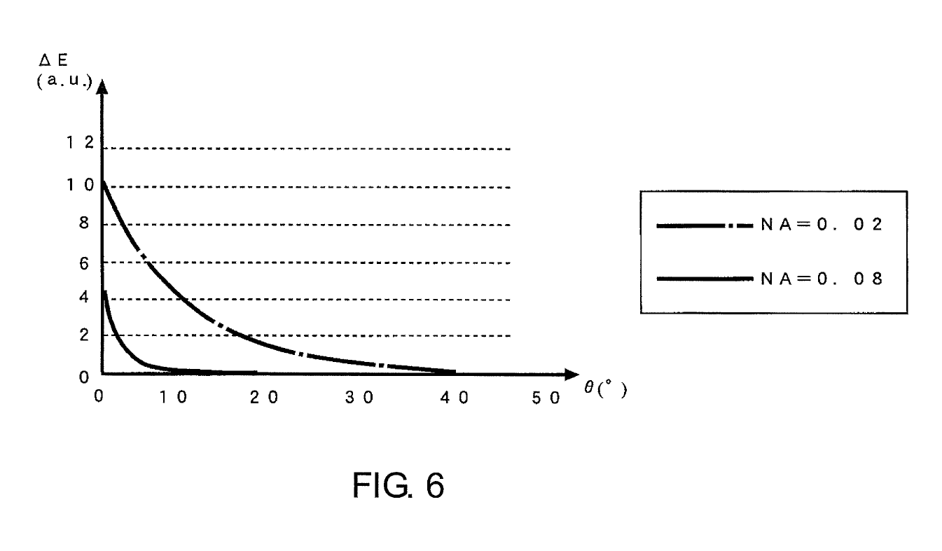

The results of the experiments derived by choosing various values of the numerical apertures NA of the condensing lens and the tilting angles ω in the measuring unit 1 will be presented below. FIG. 6 is a graph showing the stabilities of the electrical signals of the light receiving unit 24, derived at each of settings of the condensing lens 23 and the light receiving unit 24 in the measurement unit 1, according to the first embodiment. The vertical axis of FIG. 6 shows the difference value ΔE (a.u.) between the peak value and the bottom value of the electrical signals of the light receiving unit 24 measured at the corresponding numerical aperture NA and the corresponding angle of incidence θ(°). The horizontal axis of FIG. 6 shows the angle of incidence θ (°). In FIG. 6, the chain line shows the difference value ΔE when the lens with NA value of 0.02 is used as the condensing lens 23 and the solid line shows the difference value ΔE when the lens with NA value of 0.08 is used as the condensing lens 23.

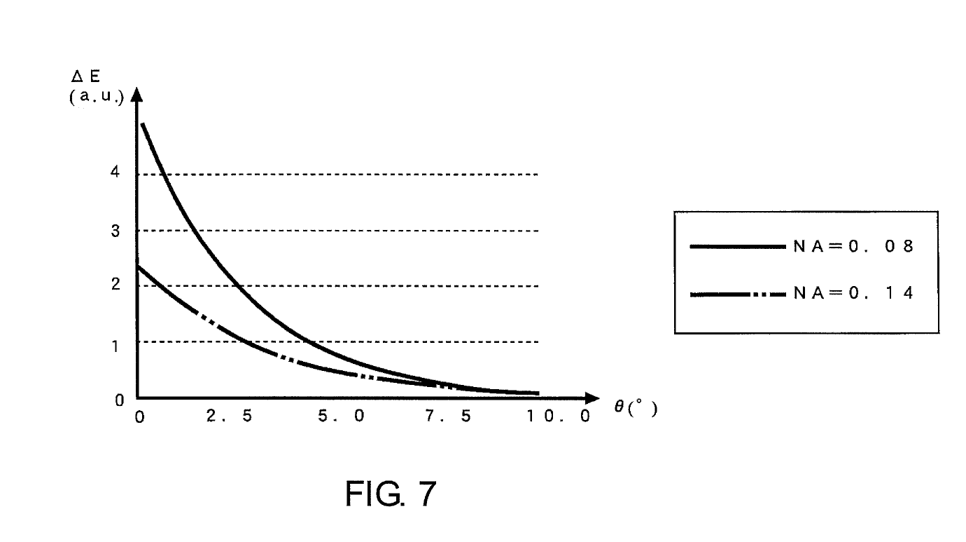

In addition, as shown in FIG. 6 and FIG. 7, it has been found that the difference value ΔE can be converged to the value extremely close to 0 when the angle of incidence is greater than or equal to 10 degrees when the value of the numerical aperture is greater than or equal to 0.08. FIG. 7 is an enlarged view of a part of FIG. 6. The vertical axis and the horizontal axis of FIG. 7 show the same parameters as those in FIG. 6. In FIG. 7, the solid line shows the difference value ΔE when the lens with NA value of 0.08 is used as the condensing lens 23 and the chain double-dashed line shows the difference value ΔE when the lens with NA value of 0.14 is used as the condensing lens 23.

As shown in FIG. 6 and FIG. 7, by setting the numerical aperture NA of the condensing lens 23 to be greater than or equal to 0.08, a more accurate electrical signal can be derived from the light receiving unit 24. Additionally, by setting the angle of incidence θ to be greater than or equal to 10 degrees, a more accurate electrical signal can be derived. Therefore, in the gas analyzing apparatus 100 comprising the measurement unit 1 in which the condensing lens 23 and the light receiving unit 24 are set in such a manner, the analysis of the sample gas Sg can be performed more accurately based on the more accurate electrical signal.

### Second Embodiment

In the above-described first embodiment, the example in which the present invention is applied to the probe-type measurement unit has been shown. However, the present invention may be applied to an open-path measurement unit. The measurement unit 2 according to the second embodiment and the gas analyzing apparatus 200 using the measurement unit 2 will be explained below. The elements that are the same as those in the above-described first embodiment are assigned to the same numerals as those in the first embodiment, and the detailed explanations are omitted.

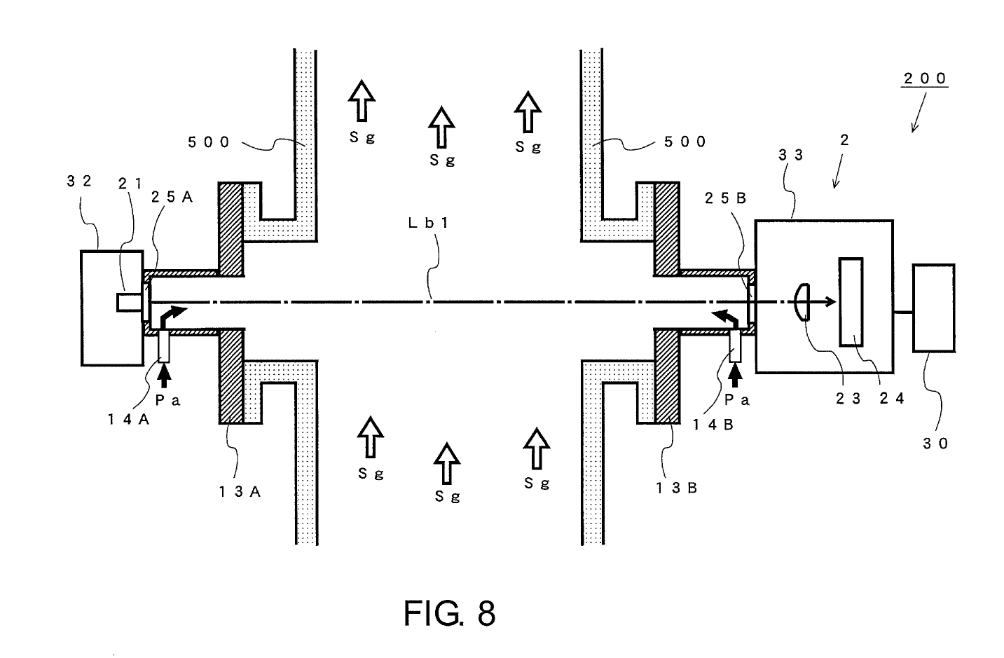

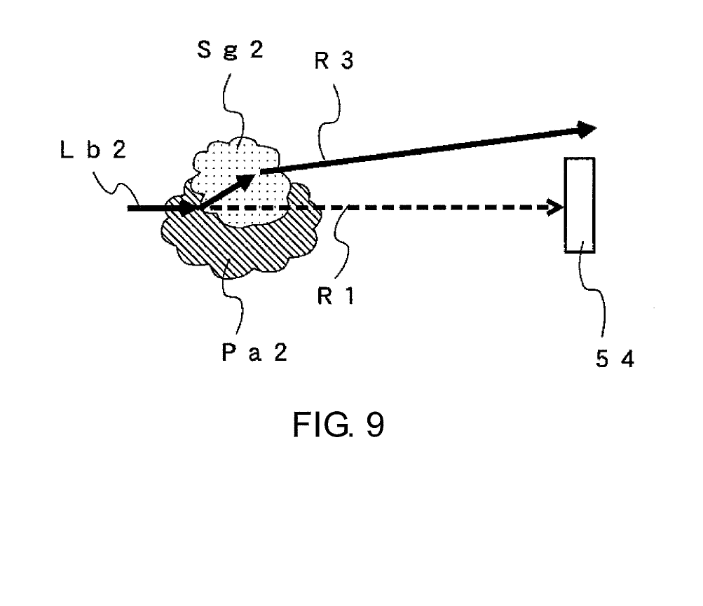

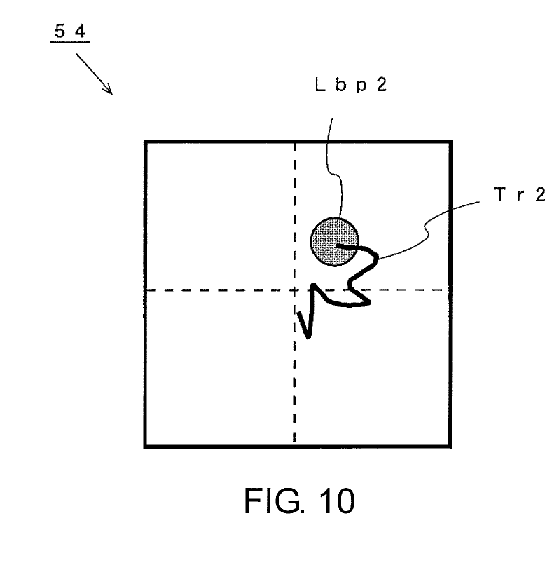

FIG. 8 is a cross-sectional view showing the inner structure of the measurement unit 2 according to the second embodiment. As shown in FIG. 8, the measurement unit 2 includes an oscillator unit 32 and a detector unit 33 that are formed separately. The oscillator unit 32 is attached at one side plane of a funnel 500. The sample gas Sg flows in the funnel 500. The detector unit 33 is attached to a different side plane of the funnel 500, so that the oscillator unit 32 and the detector unit 33 face each other.

The oscillator unit 32 includes a light emitting unit 21, an optical window 25A, a purge air introducing port 14A, and a flange 13A. The optical window 25A is arranged immediately in front of the light emitting unit 21 and the purge air introducing port 14A introduces the purge air Pa into the space that is connected to the funnel 500 immediately in front of the optical window 25A. The detector unit 33 includes a condensing lens 23, a light receiving unit 24, an optical window 25B, a purge air introducing port 14B, and a flange 13B. The condensing lens 23 is arranged immediately in front of the light receiving unit 24. The optical window 25B is arranged immediately in front of the condensing lens 23. The purge air introducing port 14B introduces the purge air Pa into the space that is connected to the funnel 500 immediately in front of the optical window 25B.

The oscillator unit 32 and the detector unit 33 are attached to the funnel 500 via the flanges 13A and 13B, respectively, while their positions are adjusted in advance, so that the measurement light Lb1 emitted by the light emitting unit 21 is emitted toward the light receiving unit 24.

With the above-described measurement unit 2, like the first embodiment, the measurement light Lb1 can be condensed properly within the light receiving plane of the light receiving unit 24 even if the measurement light Lb1 is bent due to the thermal lens effect caused by the purge air Pa and the sample gas Sg. It is also preferable in the second embodiment that the numerical aperture NA is set to be greater than or equal to 0.08 and further the angle of incidence θ is set to be greater than or equal to 10 degrees.

### INDUSTRIAL APPLICABILITY

The measurement unit and the gas analyzing apparatus according to the present invention are useful for the measurement unit, the gas analyzing apparatus, etc. that can analyze the sample gas more accurately than the conventional ones.

### EXPLANATION OF REFERENCE NUMERALS

100, 200 gas analyzing apparatus

1, 2 measurement unit

11 probe tube

12 optical unit

13 flange

14 purge air introducing port

16 purge air introducing pipe

21 light emitting unit

22 reflection mirror

23 condensing lens

24 receiving unit

30 processing apparatus

241 AR coating layer

242 InGaAs absorbing layer

243 InP wafer layer

244 package substrate

32 oscillator unit

33 detector unit

54 light receiving sensor

1. An apparatus for measuring concentrations of component gases in a sample gas, comprising:
a light emitting unit configured to emit a measurement light to the sample gas;
a light receiving unit configured to receive the measurement light on a light receiving plane;
a purge air introducing unit configured to introduce a purge air into a vicinity of at least one of the light emitting unit and the light receiving unit; and
a condensing lens arranged in an optical path and configured to condense the measurement light within the light receiving plane of the light receiving unit,
the optical path being a path of the measurement light extending from the light emitting unit to the light receiving unit,
a propagation path of the measurement light being varied due to a thermal lens effect, and
the thermal lens effect being caused by a temperature difference between the sample gas and the purge air.

2. The apparatus according to claim 1, wherein the condensing lens is arranged immediately in front of the light receiving unit,
the measurement unit further includes an optical window arranged immediately in front of the condensing lens, and configured to protect at least the condensing lens, and
the purge air introducing unit introduces the purge air immediately in front of the optical window.

3. The apparatus according to claim 1 further comprising a cylindrical probe tube having openings to introduce the sample gas into the probe tube, wherein
the purge air introducing unit introduces the purge air inside the probe tube, and
the light emitting unit is configured to emit the measurement light to the sample gas introduced inside the probe tube.

4. The apparatus according to claim 3, further comprising a reflection mirror arranged at one end portion of the probe tube, wherein:
the light emitting unit is arranged at another end portion of the probe tube, the light emitting unit being configured to emit the measurement light toward the reflection mirror, and
the light receiving unit is arranged at the another end portion of the probe tube, the light receiving unit being configured to receive the measurement light that has been reflected by the reflection mirror.

5. The apparatus according to any claim 1 wherein numerical aperture of the condensing lens is greater than or equal to 0.08.

6. The apparatus according to claim 1 wherein the light receiving unit is tilted with respect to the condensing lens such that an angle between the light receiving plane and an image formation plane of the condensing lens is greater than or equal to 10 degrees.

7. The apparatus according to claim 1 further comprising:
a processing apparatus configured to calculate concentrations of component gases in the sample gas based on a signal received from the light receiving unit.

8. An apparatus for measuring concentrations of component gases in a sample gas, comprising:
a probe tube having openings to introduce the sample gas into the probe tube;
a light emitting unit configured to emit a measurement light inside to the sample gas inside the probe tube;
a light receiving unit configured to receive the measurement light on a light receiving plane;
a purge air introducing unit configured to introduce purge air into the probe tube; and
a condensing lens arranged in an optical path in front of the light receiving unit and configured to condense the measurement light within the light receiving plane of the light receiving unit, the optical path extending from the light emitting unit to the light receiving unit, a propagation path of the measurement light being varied due to a temperature difference between the sample gas and the purge air.

9. The apparatus of claim 8 further comprising a reflection mirror positioned within the probe tube to reflect light from the light emitting unit to the light receiving unit after passing through at least a portion of the sample gas and purge air inside the probe tube.

10. The apparatus of claim 8 wherein the light receiving unit is tilted with respect to the condensing lens.

11. The apparatus of claim 8 further comprising a processing apparatus in communication with the light receiving unit and configured to calculate concentrations of component gases in the sample gas.

12. An apparatus for measuring concentrations of component gases in a sample gas, comprising:
a cylindrical probe tube having openings to introduce the sample gas into the probe tube;
a light emitting unit configured to emit a measurement light to the sample gas inside the probe tube;
a light receiving unit configured to receive the measurement light after passing through the sample gas inside the probe tube on a light receiving plane;
a purge air introducing unit configured to introduce purge air having a temperature different from a sample gas temperature into the cylindrical probe tube in a vicinity of at least one of the light emitting unit and the light receiving unit;
a reflector disposed within the cylindrical probe tube to reflect light from the light emitting unit through the sample gas inside the probe tube to the light receiving unit; and
a condensing lens arranged in an optical path of the measurement light and configured to condense the measurement light within the light receiving plane of the light receiving unit, the condensing lens having an image formation plane tilted relative to the light receiving plane;
an optical window arranged in front of the condensing lens; and
a processing apparatus configured to receive a signal from the light receiving unit and to calculate concentrations of component gases in the sample gas.

13. The apparatus of claim 12 wherein the purge air introducing unit introduces the purge air immediately in front of the optical window.

14. The apparatus of claim 12 wherein the reflector comprises a mirror arranged at one end portion of the cylindrical probe tube.

15. The apparatus of claim 12 wherein numerical aperture of the condensing lens is greater than or equal to 0.08.

16. The apparatus of claim 12 wherein the light receiving unit is tilted with respect to the condensing lens such that an angle between the light receiving plane and an image formation plane of the condensing lens is greater than or equal to 10 degrees.
